(19) United States
(12) Patent Application Publication
(10) Pub. No.: US 2014/0350337 A1
(43) Pub. Date: Nov. 27, 2014

(54) SYSTEMS, DEVICES, AND METHODS FOR PROVIDING INSERTABLE ROBOTIC SENSORY AND MANIPULATION PLATFORMS FOR SINGLE PORT SURGERY

(71) Applicant: THE TRUSTEES OF COLUMBIA UNIVERSITY IN THE CITY OF NEW YORK, New York, NY (US)

(72) Inventors: Nabil SIMAAN, Nashville, TN (US); Kai XU, New York, NY (US); Roger GOLDMAN, New York, NY (US); Peter ALLEN, Pleasantville, NY (US); Dennis FOWLER, New York, NY (US); Jienan DING, Ny, NY (US)

(73) Assignee: THE TRUSTEES OF COLUMBIA UNIVERSITY IN THE CITY OF NEW YORK, New York, NY (US)

(21) Appl. No.: 14/222,232

(22) Filed: Mar. 21, 2014

Related U.S. Application Data
(63) Continuation of application No. 13/063,615, filed on May 9, 2011, now abandoned.
(60) Provisional application No. 61/103,415, filed on Oct. 7, 2008.

(51) Int. Cl. A61B 19/00; A61B 1/005; A61B 1/00; A61B 17/00; A61B 1/05

(52) U.S. Cl. CPC A61B 19/2203; A61B 17/00234; A61B 1/051; A61B 1/00193; A61B 1/0055; A61B 2019/2211; A61B 2019/2215; A61B 2019/2238; A61B 2017/00296; A61B 2019/2223
USPC 600/106

(57) ABSTRACT

The present disclosure relates to systems, devices, and methods for providing foldable, insertable robotic sensory and manipulation platforms for single port surgery. The device is referred to herein as an Insertable Robotic Effector Platform (IREP). The IREP provides a self-deployable insertable device that provides stereo visual feedback upon insertion, implements a backbone structure having a primary backbone and four secondary backbones for each of the robotic arms, and implements a radial expansion mechanism that can separate the robotic arms. All of these elements together provide an anthropomorphic endoscopic device.

### CROSS REFERENCE TO RELATED APPLICATIONS

This application is a continuation of application Ser. No. 13/063,615, filed on May 9, 2011, which is a U.S. National Phase application under 35 U.S.C. §371 of International Patent Application No. PCT/US2009/059827, filed on Oct. 7, 2009, which claims the benefit under 35 U.S.C. §119(e) of U.S. Provisional Application No. 61/103,415 filed on Oct. 7, 2008, each of which is hereby incorporated by reference in its entirety.

### STATEMENT REGARDING FEDERALLY SPONSORED RESEARCH

The present invention was supported by grants from the National Institute of Health grant number: 5R21EB007779-02. The U.S. Government may have certain rights to the present invention.

### FIELD OF THE INVENTION

The present invention relates to devices, systems and surgical techniques for minimally invasive surgery and more particularly to minimally invasive devices, systems and surgical techniques/methods associated with treatment, biopsy and the like of body cavities.

### BACKGROUND

Laparoscopic and other minimally invasive surgeries have successfully reduced patients' post operative pain, complications, hospitalization time and improved cosmesis. See D. J. Deziel, K. W. Millikan, S. G. Economou, M. A. Doolas, S. -T. Ko, and M. C. Airan, “Complications of Laparoscopic Cholecystectomy: A National Survey of 4,292 Hospitals and an Analysis of 77,604 Cases,” The American Journal of Surgery, vol. 165, No. 1, pp. 9-14, January 1993; and M. J. Mack, “Minimally Invasive and Robotic Surgery,” The Journal of the American Medical Association, vol. 285, No. 5, pp. 568-572, Feb. 7, 2001. During most laparoscopic procedures, two or more incisions are used for surgical instruments, visualization, and insufflation. See E. Berber, K. L. Engle, A. Garland, A. String, A. Foroutani, J. M. Pearl, and A. E. Siperstein, “A Critical Analysis of Intraoperative Time Utilization in Laparoscopic Cholecystectomy,” Surgical Endoscopy, vol. 15, No. 2, pp. 161-165, 2004. Before Natural Orifice Transluminal Endoscopic Surgery (N.O.T.E.S), which eliminates all skin incisions, can be widely applied to broader procedures, population researchers and surgeons may focus on single port access (SPA) surgeries which reduce the number of skin incisions to one and therefore generate a better outcome than traditional laparoscopic procedures.

Most existing robotic surgical systems are designed for minimally invasive laparoscopic procedures. Although robotic assistance has greatly enhanced surgeons' capabilities in performing standardized laparoscopic techniques, these existing robotic systems are not suitable for SPA surgeries due to the large size of their instruments and lack of overarching and collision avoidance among its multiple arms. Therefore, SPA surgeries are currently limited to just a few academic centers using specifically modified laparoscopic tools (such as RealHand™ (Novare Surgical Systems, Inc., Cupertino, Calif.)).

### SUMMARY

The present disclosure relates to systems, devices, and methods for providing foldable, insertable robotic sensory and manipulation platforms for single port surgery. The device is referred to herein as an Insertable Robotic Effector Platform (IREP). The IREP provides a self-deployable insertable device that provides stereo visual feedback upon insertion, implements a backbone structure having a primary backbone and four secondary backbones for each of the robotic arms, and implements a radial expansion mechanism that can separate the robotic arms. All of these elements together provide an anthropomorphic endoscopic device.

In one aspect, the IREP provides endoscopic imaging and distal dexterity enhancement. The IREP robot includes two five-degree of freedom snake-like continuum robots, two two-degree of freedom parallelogram mechanisms, and one three-degree of freedom stereo vision module. The IREP can be used in abdominal SPA procedures, such as cholecystectomy, appendectomy, liver resection, among others. The IREP can fit through a small skin incision while providing vision feedback to guide insertion and deployment of two dexterous arms with a controllable stereo vision module.

### BRIEF DESCRIPTION OF THE DRAWINGS

For a more complete understanding of various embodiments of the present disclosure, reference is now made to the following descriptions taken in connection with the accompanying drawings in which:

FIG. 1A depicts a system overview of the IREP Robot in a folded configuration, according to one or more embodiments of the present disclosure;

FIG. 1B depicts methods of detachable actuation transmission using wire actuation and push-pull super-elastic NiTi backbones;

FIG. 2 depicts a system overview of the IREP Robot in a working configuration, according to one or more embodiments of the present disclosure;

FIGS. 3A-3F depict an image sequence showing the deployment of the IREP robot, according to one or more embodiments of the present disclosure;

FIG. 4A is a depiction of the camera module of the IREP robot, according to one or more embodiments of the present disclosure;

FIG. 4B is an exploded view of the camera module shown in FIG. 4, according to one or more embodiments of the present disclosure;

FIGS. 5 is a depiction of a single dexterous arm of the IREP, according to one or more embodiments of the present disclosure;

FIG. 6 is a depiction of a backbone structure of the IREP Robot, according to one or more embodiments of the present disclosure;

FIG. 7A is a depiction of a parallelogram actuation unit of the IREP Robot, according to one or more embodiments of the present disclosure;

FIG. 7B is another depiction of the parallelogram actuation unit of the IREP Robot, according to one or more embodiments of the present disclosure;

FIG. 8 is a depiction of the translational workspaces of the right arm, left arm and overlapping areas, according to one or more embodiments of the present disclosure;

FIG. 9 is a depiction of a gripper of the IREP Robot, according to one or more embodiments of the present disclosure;

FIG. 10 is a depiction of gripper teeth, showing different teeth for different suture sizes, according to one or more embodiments of the present disclosure;

FIG. 11 is a depiction of two connected slots for both high end gripping force and wide open angle of the IREP Robot, according to one or more embodiments of the present disclosure;

FIG. 12 is a graph depicting actuation force with respect to jaw angle of the IREP Robot, according to one or more embodiments of the present disclosure;

FIG. 13A is a depiction of a wrist of the IREP Robot, according to one or more embodiments of the present disclosure;

FIG. 13B is an exploded view of the wrist shown in FIG. 13A;

FIG. 14 depicts a dual arm suturing capability of the IREP Robot, according to one or more embodiments of the present disclosure;

FIGS. 15A-F depicts a suturing simulation using the IREP Robot, according to one or more embodiments of the present disclosure; and

FIG. 16 is a block diagram of the control system architecture for the IREP Robot, according to one or more embodiments of the present disclosure.

### DETAILED DESCRIPTION

The present disclosure relates to a foldable, insertable robotic surgical device and its method of use. The IREP robot includes two five-degree of freedom snake-like continuum robots, two two-degree of freedom radial extension mechanisms, and one three-degree of freedom stereo vision module.

Robot-assisted SPA surgery desirably has the following capabilities:
i) the robot has a folded configuration for it to pass through a single small skin incision,
ii) the robot is self deployable into a working configuration,
iii) the target organs and their related tissues (such as gallbladder, hepatic tissues, pancreas, etc.) can be manipulated with enough precision and force,
iv) the translational workspace is bigger than 50 mm×50 mm×50 mm (e.g., the size of the target organs),
v) the robot has a stereo vision unit for depth perception and tool tracking, and
vi) the illumination device is integrated into the robot.

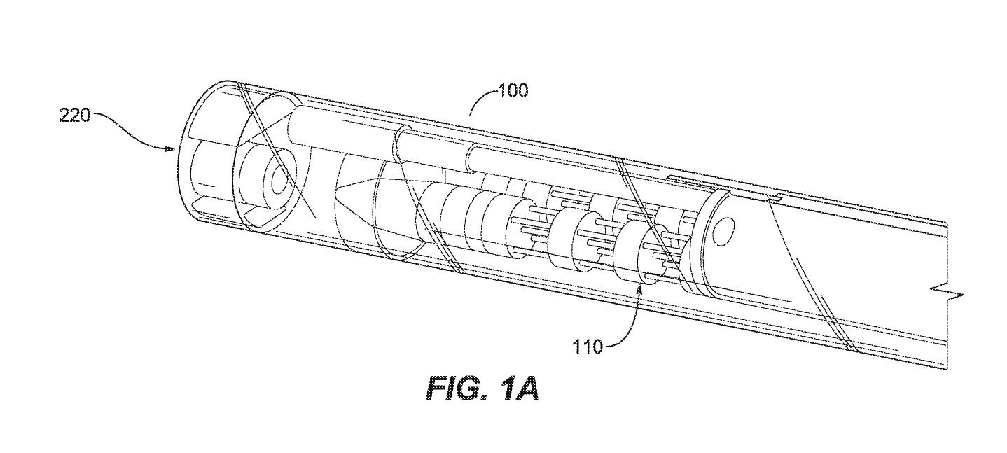

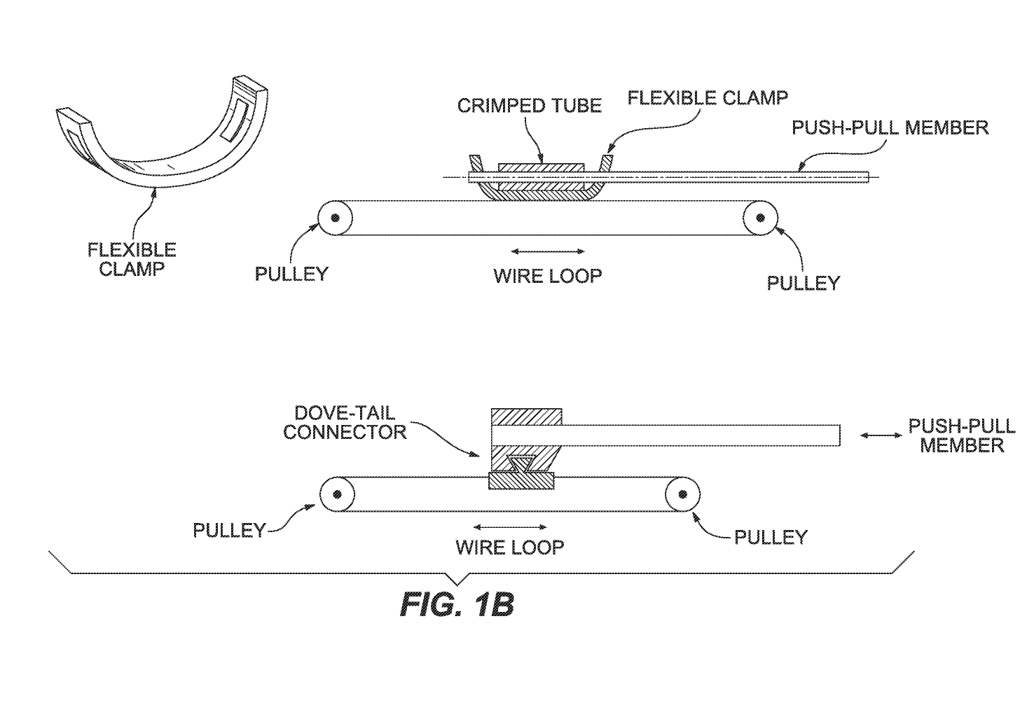

FIG. 1 depicts a system overview of the IREP Robot 100 in a folded configuration, according to one or more embodiments of the present disclosure. The IREP robot of FIG. 1 demonstrates the features and capabilities for SPA surgery. When it is in its folded configuration (as illustrated in FIG. 1), it can be deployed into the abdomen through a small, e.g., Ø15 mm skin incision, while using its forward-looking stereo vision module 220 to guide surgeons through the insertion phase. The IREP Robot 100 includes an elongated lumen 110 that encloses the various elements of the robot. The lumen 110 can be constructed from the following materials: stainless steel, anodized aluminum, titanium, or molded plastic. In some embodiments, the lumen has an outer diameter of 15 mm.

In some embodiments, the outer diameter of the IREP in folded configuration is 15 mm. In some embodiments, the lumen 110 is rigid. This dimension is currently limited by the Ø6.5 mm diameter of the CCD cameras (Model Number, CSH-1.4-V4-END-R1 from NET, Inc.) used in the stereo vision module 120. The two cameras are placed next to one another in order to simulate the positioning of human eyes. Placing the cameras axially displaced along the axis of the IREP will make the IREP's insertable portion too long to allow its deployment inside a small cavity. Placing the cameras in parallel will take a diameter of 13 mm, which leaves space for protective covers. Since in a Ø20 mm incision is available for transumbilical laparoscopic procedures, a diameter of 15 mm of the IREP is acceptable. There are smaller cameras that suffer from image distortion and sensitivity to lighting conditions that make 3D stereo-vision tool tracking less accurate; however, it is expected that improvements in cameras would permit incorporation of smaller cameras with resulting smaller outer diameter to the device. The other limitation of the outer diameter can come from the required diameter for the dexterous snake arms (continuum robots) in order to support forces of interaction typical to abdominal applications.

In some embodiments, a passively flexible central lumen may be constructed using wire actuated designs wherein the superstructure of the lumen may be made of a flexible structure that passively bends to accommodate the anatomy and provides passage for the actuation wires of the IREP. The flexible lumen may be made of polymer elastomers that are superelastic tube micro-machined to provide flexure hinges, or any other serial linkage design.

When using a passively flexible central lumen, the actuation of the IREP may still be achieved using a connection method between the push-pull components of the IREP and the actuation wires as shown in FIG. 1B. The distal and proximal ends of the flexible lumen can be modified to include small pulleys used to tension actuation wire loops. Through actuation of these wire loops, all the components of the IREP can be actuated through fast clamping attachments such as the flexible clamp or the dove-tail connector of FIG. 1B.

Actively actuated central lumens may be designed using, for example, wire-actuated articulated designs such as (Degani et al. 2006) and (Gottumukkala et al. 2004). These designs allow alternating relaxation and locking of a passive lumen in order to allow it to follow the shape of the anatomy. Regardless of the technology used to achieve a passively steerable lumen, the IREP may still be actuated using the same approach as in passively flexible central lumens.

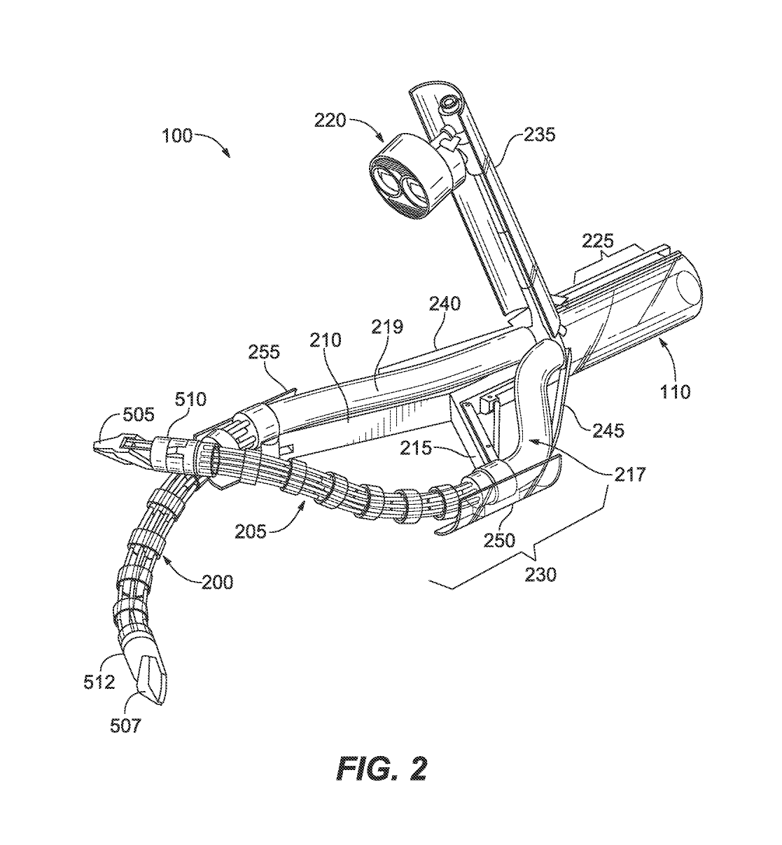

The IREP can unfold itself into a working configuration to perform SPA procedures, as shown in FIG. 2. FIG. 2 depicts a system overview of the IREP Robot in a working configuration, according to one or more embodiments of the present disclosure. The IREP robot 100 consists of two snakelike continuum robots 200, 205, two radial extension mechanisms 210, 215, two flexible stems, 217, 219, and one 3D stereo vision module 220, wherein the vision module 220 is comprised of two CCD cameras for stereo visual feedback. The two dexterous snake-like arms are equipped with distal wrists 510, 512 and grippers 505, 507.

When in a deployed configuration, as shown in FIG. 2, the proximal portion of the lumen 225 remains intact, while the distal portion of the lumen 230 separates into multiple segments. These segments can include a top semi-circular segment 235 that overlays the stereo vision module 220. The bottom semi-circular portion of the lumen can be divided in four segments. Two quarter-circular segments 240, 245, for example, each half the length of the top segment 235, extend from the proximate portion of the lumen 225 along each of flexible stems 217, 219. The other two quarter-circular segments 250, 255 are located at the joint between the flexible stems 217, 219 and the continuum robots 200, 205. The segmentation of the lumen provides a compact deployment mechanism. Instead of having to use an overtube to protect the robot, the thin segmented lumen reduces the set up time of the procedure and the size of the incision. The segmented sections also prevent the opened lumen segments from interfering with the procedure.

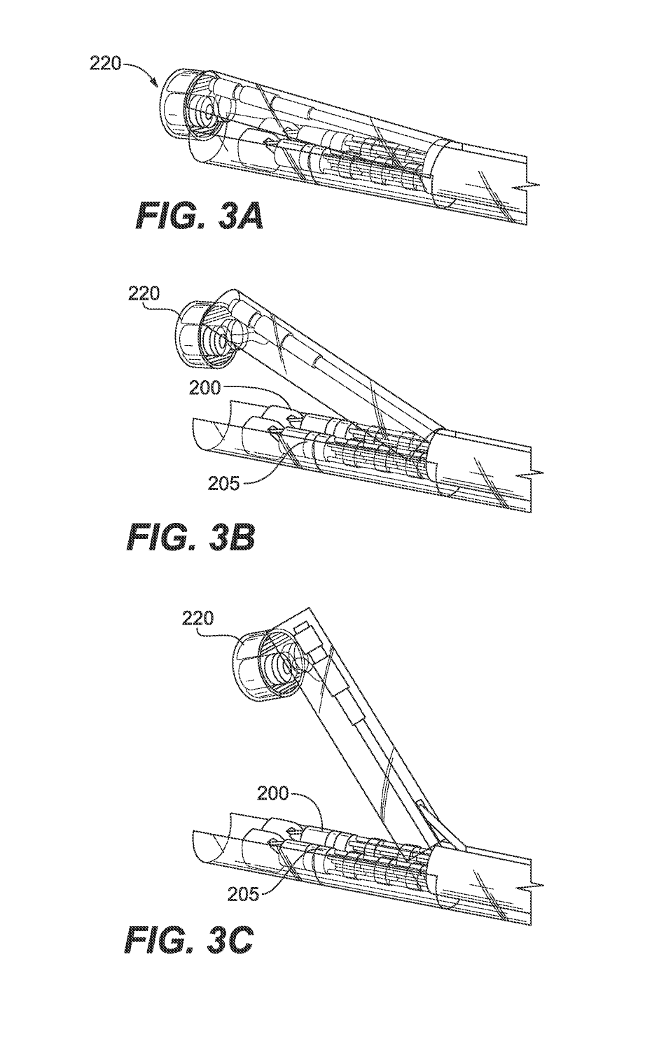

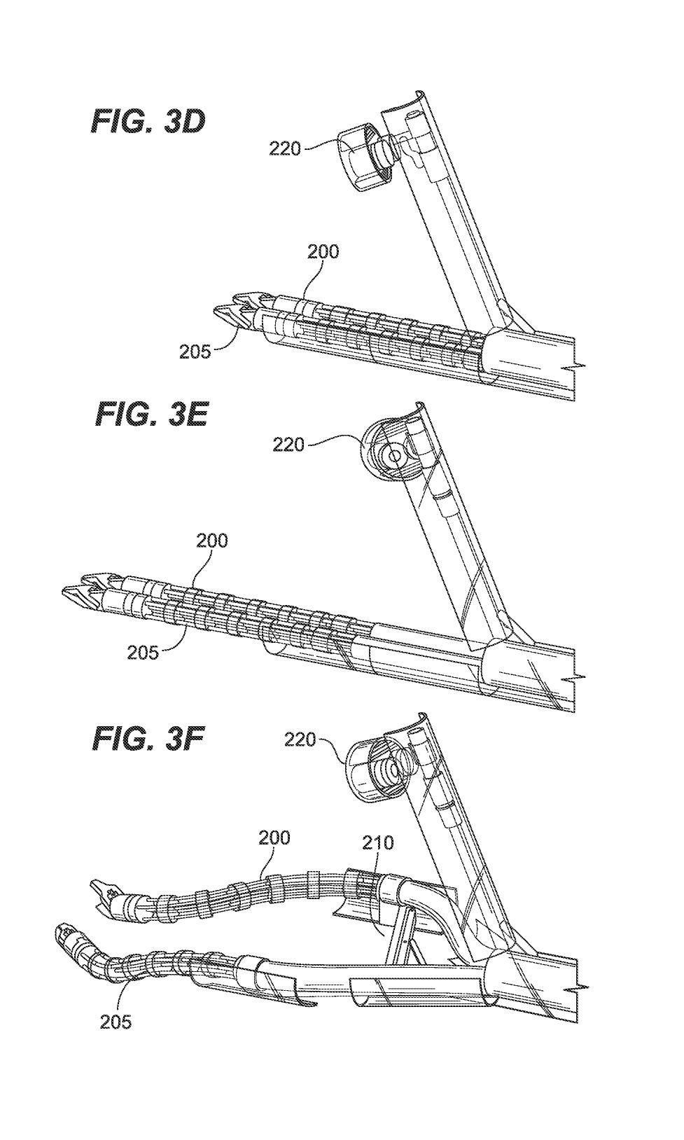

FIGS. 3A-3F depict an image sequence showing the deployment of the IREP robot, according to one or more embodiments of the present disclosure. The IREP robot can be inserted into patient's abdominal cavity in its folded configuration and then the device can unfold itself into a working or deployed configuration. FIG. 3A depicts the stereo vision module 220 separating from the lumen 110. FIGS. 3B and 3C shows further separation from the vision module 220 and the lumen 110 and exposes the continuum robot arms 200, 205. FIGS. 3D and 3E show the continuum robot arms 200, 205 extending along the longitudinal axis of the lumen. FIG. 3F shows the final deployed configuration where the radial extension devices 210, 215 (also referred to herein as parallelogram devices) have radially separated the continuum robot arms 200, 205 from each other.

The IREP has a plurality of actuators, for example, 21 actuators, that drive its two dexterous or continuum arms, vision module, and two five-bar (radial extension) mechanisms that allow self deployment of the dexterous arms and adjustment of the distance between the bases of the two arms. The IREP can actively change from insertion to working configuration while providing uninterrupted 3D stereo vision feedback to the user. During insertion, the IREP is folded into a cylindrical configuration with a diameter of about 15 mm (FIG. 1). Insertion into the patient abdomen can be carried out using a trocar at the umbilicus. After insertion, the IREP deploys two dexterous snake-like arms equipped with distal wrists 510, 512 and grippers 505, 507. A third arm is also deployed with a 3D vision module comprised of two CCD cameras for stereo visual feedback. Each dexterous arm includes a four degree of freedom two-segment continuum snake-like robot, a single degree of freedom wrist, and a gripper. When supported on a five-bar radial extension mechanism 215, 210, the robot arm can provide seven degrees of freedom of motion using its eight actuated joints and the additional actuated joint available for its gripper.

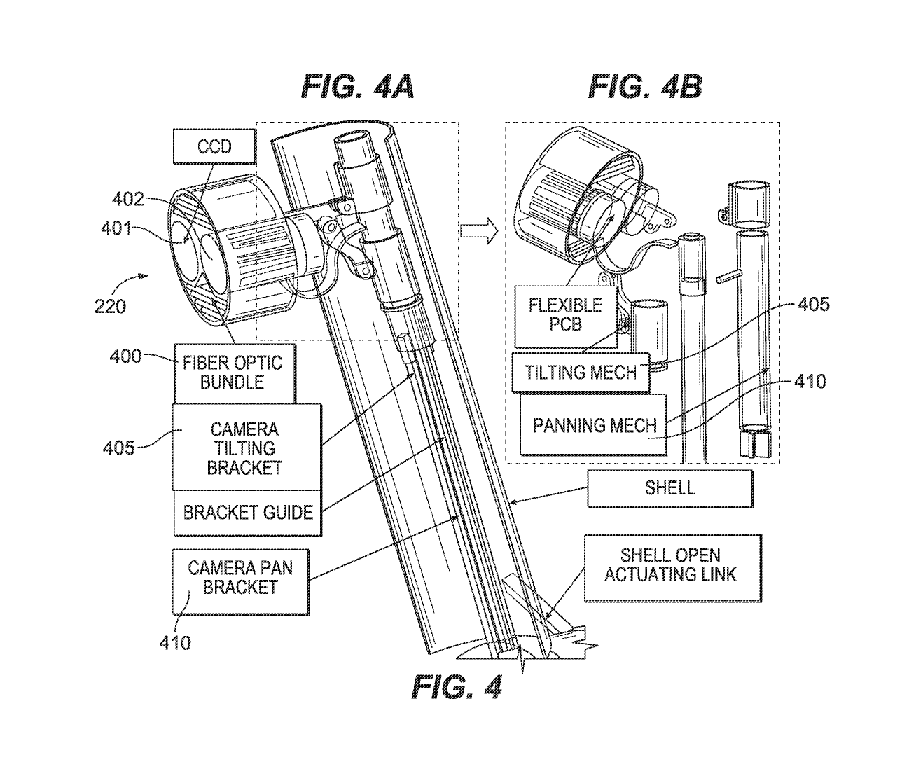

FIG. 4A is a depiction of the stereo vision camera module 220 of the IREP robot, according to one or more embodiments of the present disclosure. FIG. 4B is an exploded view of the camera module of FIG. 4A. The stereo vision module 220 has a pair of CCD cameras 401, 402 for depth perception as well as surgical tool tracking The camera module has three degrees of freedoms for pan (using the panning mechanism 410), tilt (using the tilting mechanism 405), and zoom adjustments. A light source using optic fiber bundles 400 is also integrated into the camera module. The device can close to a Ø15 mm cross section. The camera housing encloses two camera units consisting of housing and two degree of freedom actuated joint that allows panning and tilting the housing in two directions as shown in FIG. 4. The camera module 220 is supported on one side of the lumen and can be controlled independently of the lumen opening. The control mechanism for the camera module uses a slider-crank mechanism for control of the tilt angle. Actuation of the tilting mechanism is achieved via a thin NiTi superelastic wire that is supported in a dedicated channel in the central lumen 110 such that it can withstand compressive and tensile forces (push-pull actuation). The panning mechanism is used to control the panning angle of the camera module. This mechanism is also actuated by a NiTi wire in push pull actuation. The axial translation of the actuation wire translates a pin in a helical slot in the panning mechanism tube. This causes the panning mechanism to rotate about its longitudinal axis, which provides the panning degree of freedom. The electronic signals to the camera module are transmitted using a flexible printed circuit board (PCB). The angle of the outer shell carrying the camera module and its actuation mechanisms is controlled via a slider-crank mechanism in which the shell actuating link acts as the pushrod and the shell acts as the crank. This shell actuating link is actuated by a link that translates prismatically inside the central lumen.

The camera system is used as follows:
1) it provides the surgeon with a means for monitoring and controlling the movements of the robotic arms;
2) it provides a means for light-based imaging that the surgeon can use for identifications of pathologies;
3) in the folded state of the robot of FIG. 1 the cameras point forward in the direction of insertion and help the surgeon see the various stages of the insertion of the robot into the anatomy.

One advantage of the proposed design in FIG. 4 is that it offers an anthropomorphic stereo-vision and manipulation setup that mimics the human anatomy in which the field of focus of the eyes is located between the two manipulation arms. The vision module has two integrated stereo vision CCD cameras with a baseline of 7.6 mm. These CCD cameras are attached to a controllable shell with adjustable pan and tilt for increased visual field. This camera-between-hands arrangement provides an anthropomorphic and intuitive image to surgeons who are used to operating on surgical sites located between their own arms. The pan and tilt angles of the stereo vision cameras are controllable by a pull-push mechanism that allows instrument tracking During insertion, the robotic platform is folded and its stereo vision module points forward in order to provide vision feedback to the surgeon.

To integrate a stereo vision module for tracking surgical tool tip's movement, the baseline between the two CCD cameras can be maximized for improved tracking precision.

The system configuration is shown FIG. 1, where the two CCD cameras are packed together. A fixed baseline simplifies calibration. Initial simulation showed an accuracy of approximate 0.16 mm. In addition, the central stem has available a cross sectional area of 36 mm² in for passing through optic fiber bundles for illuminations.

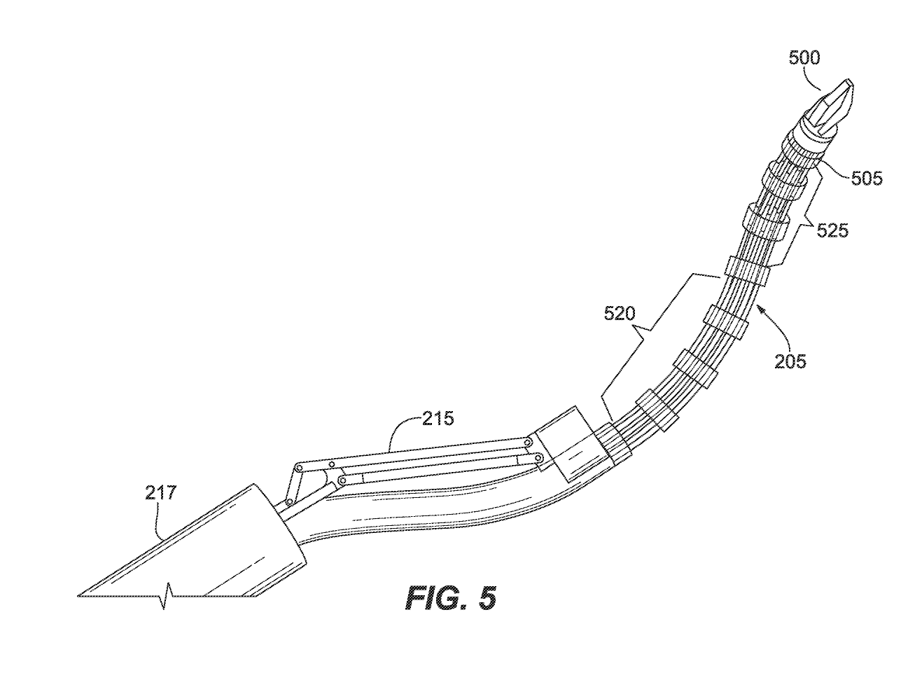

FIG. 5 is a depiction of a single dexterous arm of the IREP, according to one or more embodiments of the present disclosure. Each dexterous arm includes at least four components:
i) a gripper 500,
ii) a one-Degree of freedom wrist 505,
iii) a four-Degree of freedom continuum robot/snake arm 205,
iv) a radial extension mechanism 215 and
v) a flexible stem 217.

Each single dexterous arm acts as a surgical telemanipulation slave for dual arm interventions and delivery of sensors (e.g. ultrasound probe) or energy sources (e.g. cautery). During SPA procedures, each of the arms of the IREP robot can be independently pulled out and replaced with another arm equipped with different surgical end effectors. As shown in FIG. 5, the continuum robot can include two structures or segments: a first structure 520 and a second structure 525. These structures are referred to as backbones and are discussed in more detail below.

One purpose of the dual arm device of FIG. 5, for example, is to provide dexterous tool manipulation. Some embodiments of the design in FIG. 5 can be combined with, for example, U.S. patent application Ser. No. 10/850,821, filed May 21, 2004 which is hereby incorporated by reference herein in its entirety. The '821 application discloses devices, systems, and methods for minimally invasive surgery of the throat and other portions of the mammalian body. The '821 discloses a dexterous arm having a primary backbone and three secondary backbones.

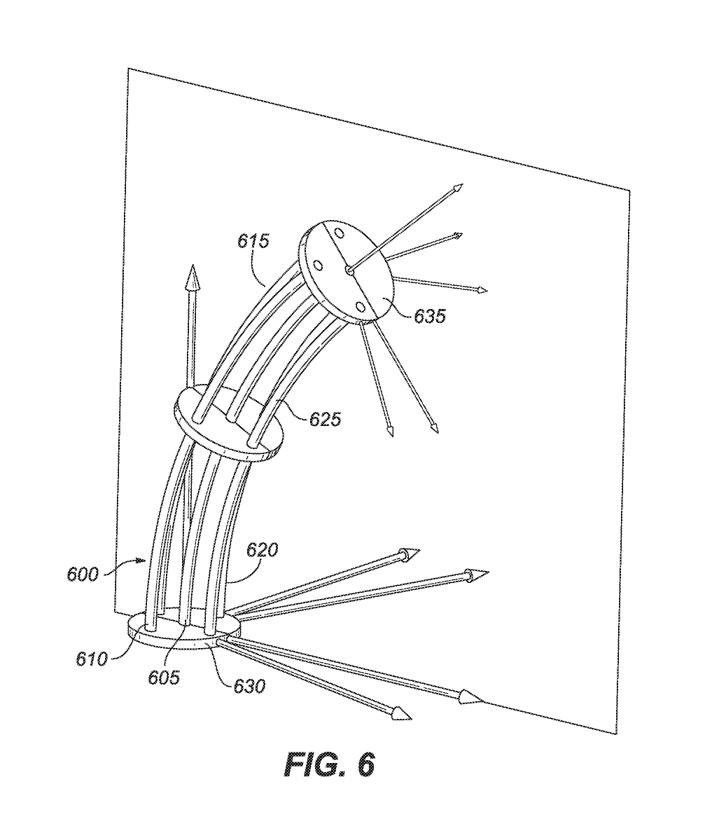

FIG. 6 is a depiction of a backbone structure 700 of the IREP Robot, according to one or more embodiments of the present disclosure. The present design uses one central super-elastic backbone 705 surrounded by four secondary superelastic tubular backbones 610, 615, 620 and 625. The backbones are connected through a series of disks, including a base disk 630, an end disk 635 and one or more spacer disks 640. While one spacer disk is shown in FIG. 6, a plurality of spacer disks can be used, depending on the size of the backbone structure. Four identical secondary backbones are equidistant from each other and from the primary backbone. The secondary backbones are only attached to the end disk and can slide in appropriately toleranced holes in the base disk and in the spacer disks. The two degree of freedom bending motion of this continuum segment is achieved through simultaneous differential actuation of the four secondary backbones. Each primary of secondary backbone can be composed of nickel titanium (NiTi) wires, cylinders or concentric cylinders. The backbones of the first and the second segments (shown in FIG. 2) are concentric NiTi super-elastic tubes with outer and inner diameters of 0.90×0.76 mm and 0.64×0.51 mm. The disks each can have a diameter of about 6.4 mm and a height of about 3.2 mm. The disks can be made from stainless steel. The diameter can be between 4.0-6.4 mm and height between 3.2-1.6 mm.

In some embodiments, two or more backbone structures can be stacked on top of each other to form elongated backbone structures with a higher degree of freedom. In one embodiment, the continuum arm is composed of two backbone structures to form the four-Degree of freedom continuum snake arm. Each structure consists of several super-elastic NiTi tubes as backbones and several disks. For example, in FIG. 5, the continuum arm can include a first structure 520 and a second structure 525. FIG. 6 shows one segment, where one primary backbone is centrally located and is attached to the base disk and the end disk.

The payload of the four degree of freedom continuum NiTi snake continuum arms determines the payload of the entire IREP robot since it is the weakest portion of the IREP robot. For this reason, the Ø6.4 mm diameter of the four-Degree of freedom continuum snake arm was maximized to use all available space in folded configuration. The diameters of the backbones were chosen to be Ø0.90 mm for the first segments of the continuum snakes and Ø0.64 mm for the distal segments. All backbones are made from super-elastic NiTi tubes to provide channels for actuation of the gripper and the wrist, suction, cautery, light, and delivery of wiring for sensors.

Previous works demonstrated that continuum snake-like robots as in FIG. 5 can serve as distal dexterity tools for enabling complicated surgical tasks such as suturing and knot tying in confined spaces. The proven dexterity plus the scalability and load-carrying capability of this type of continuum robots make it an ideal choice for the IREP robot's arms. Furthermore, its intrinsic force sensing capability developed in allows equipping the IREP robot with force sensing capabilities. For details of the force sensing capabilities, please see related application no. PCT/US09/032068, entitled, SYSTEMS AND METHODS FOR FORCE SENSING IN A ROBOT, the entire contents of which are hereby incorporated by reference.

The choice of continuum flexible robots using NiTi backbones was motivated by the inherent safety of flexible robots in manipulating organs, the enhanced miniaturization of these arms.

All these controlled joints can be actuated by NiTi tubes or stainless steel rods in push-pull mode. The actuation unit will remain outside patient's body. This configuration simplifies the design of the actuation unit for the snakes because opposing secondary backbones have to be pushed and pulled on in the same amount. Two of the secondary backbones are used for delivering wire actuation for the writs. The central backbone is used for delivering actuation for the gripper by using a superelastic wire in pushing mode. The two remaining backbones may be used for delivering other sources of energy or for sensory data.

The advantage of the five backbone design is in the simplicity of actuation since each backbone can be pulled on while the other radially-opposing backbone can be pushed by the same amount. This modification eliminates the need for software kinematic coupling between opposing backbones—a feature that simplifies deployment and homing of these robots. The wrist is a wire-driven joint that allows independent rotation of the gripper about its longitudinal axis, therefore adding dexterity critical to suturing tasks in confined spaces. While it is possible to provide rotation about the axis of the gripper by using the continuum robots as a constant velocity joint through careful coordination of actuation of all backbones, the use of an independent wrist simplifies the control and improves dexterity.

Since the two snake-like continuum robots are deployed through the IREP's Ø15 mm central stem, their direct implementation will not provide enough overlapped translational workspace. For this reason, two radial extension mechanisms, also referred to as parallelogram mechanisms, are included to control the position of the bases of the snake-like continuum robots. Translational workspace of the single four-degree of freedom continuum snakelike robot used in the arms of the IREP in FIG. 2.

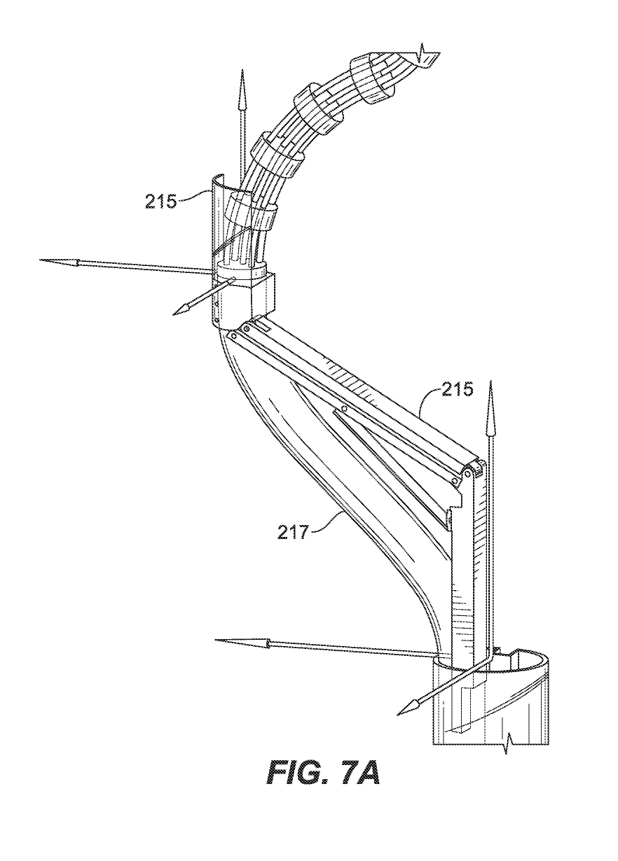

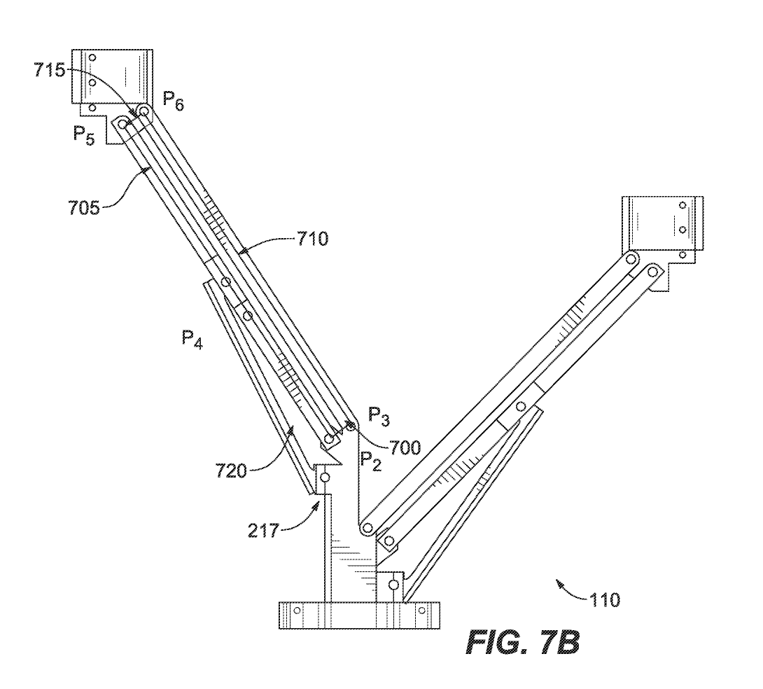

FIG. 7 is a depiction of a radial extension structure unit of the IREP Robot, according to one or more embodiments of the present disclosure. Each radial extension structure 215 has two degree of freedoms for a translational placement of the snake-like continuum robot 215. The flexible stem 217 will be independently fed in and out to comply with the radial extension structure's motion. The radial extension structure serves at least two purposes:
i) retracting the snake arms into the shell in a closed configuration (FIG. 1), and
ii) changing the distance between the base of each arm to allow for dual-arm end effector triangulation (FIG. 3E).

The radial extension structures also help in avoiding dexterity deficiencies due to “sword fighting” of the instruments. In some embodiments, the radial extension structures can be a five bar parallelogram mechanism, as shown in FIG. 7B. A shown in FIG. 7B, the five bar mechanism includes a first bar 700 between points P₂ and P₃, a second bar 705 between points P₂ and P₅, a third bar 710 between points P₃ and P₆, a fourth bar 715 between points P₅ and P₆, and a fifth bar 720 between points P₁ and P₄. All of the bars in the parallelogram mechanism can be made of stainless steel. This embodiment of the radial extension mechanism is called a parallelogram mechanism because of the parallelogram formed by points P₂, P₃, P₆, and P₅. The first bar 700 and the fourth bar 715 remain at the same orientation with respect to each other while the parallelogram mechanism is moved. The dimensions of the bars can be as follow: the first bar 700 can be about 2.3 mm, the second bar 705 can be about 35 mm, the third bar 710 can be about 35 mm, the fourth bar 715 can be about 2.3 mm, and the fifth bar 720 can be about 20 mm. The five bar mechanism is actuated by two push-pull members located in the base of the flexible stem 217. The push-pull members in the flexible stem 217 move the fifth bar 720 relative to the first bar 700, second bar 705, third bar 710 and fourth bar 715, which rotates the parallelogram mechanism radially from the lumen 110. This structure provides two degrees of freedom. These two degrees of freedom yield planar motion of the base of the snake while restricting the orientation of the base disk to be parallel with the end of the flexible stem 217.

In an embodiment of the system of FIG. 1 where the central lumen is rigid the actuation members of the parallelograms may be rigid strips actuated in push-pull mode. In an embodiment in which the central lumen of the system of FIG. 1 is flexible, the actuation of the five-bars may be achieved by wire actuation, or through flexible passively articulated linkage actuated by push-pull actuation. The wire-actuation mechanism for the case where the central lumen is flexible is as shown in FIG. 1B. Referring to FIG. 1B, it is shown that a closed-loop wire actuation mechanism is used to axially translate a flexible clamp or a dove-tail connector that is used to connect to the superelastic NiTi backbones of the continuum robots. In another embodiment, a passively articulated linkage is used to actuate the backbones of the continuum robots. The passively articulated mechanism is composed from serially connected linkage arms with passive joints connecting them. Axial transmission of load is possible as long as an outer external sheath is present to support this linkage. The function of the outer support sheath is provided by the outer flexible lumen.

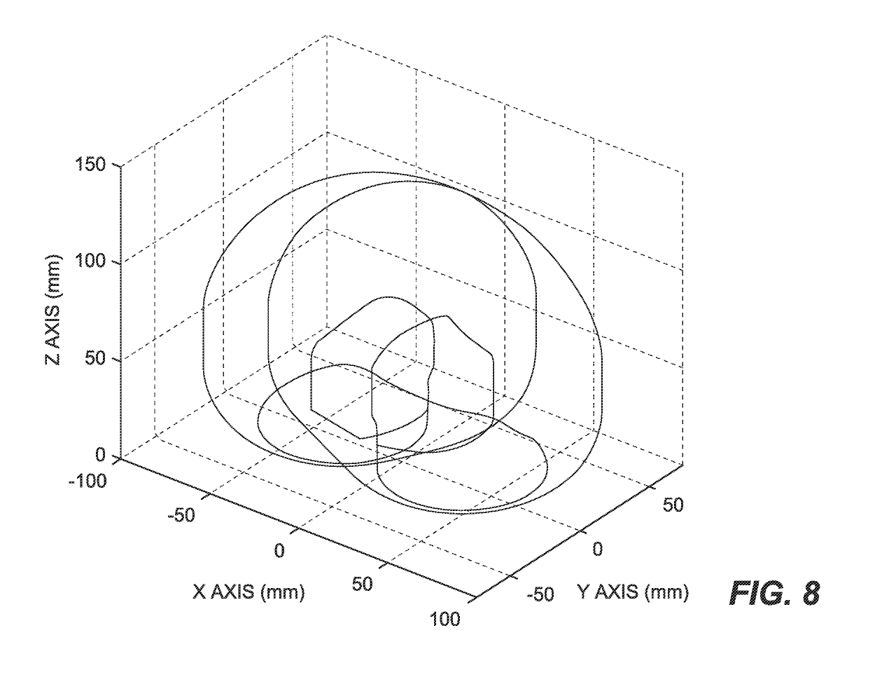

Combining the workspace of the snake-like continuum robot and that of the parallelogram mechanism, the translational workspace of the dual-arm IREP robot is plotted in FIG. 8. The figure shows that the final design fulfills the workspace requirement. When the parallelogram mechanism is actuated, the flexible stem will be fed through the central stem by the external actuation system. Thus, through the use of the radial extension mechanism, the effective workspace of the IREP is increased.

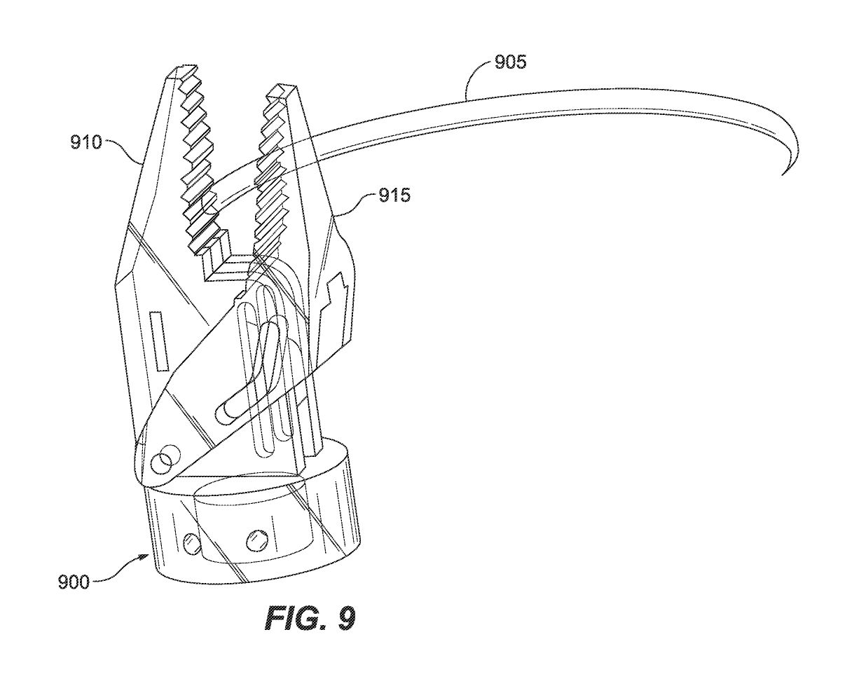

FIG. 9 is a depiction of a gripper 900 of the IREP Robot, according to one or more embodiments of the present disclosure. The gripper is attached to the wrist. The gripper includes a first opposable end piece 910 and a second opposable end piece 915. To stabilize a suture 905, the gripper is expected to provide around 40N gripping force. The gripper design then has two requirements:
i) the gripper should guarantee 40N gripping force with minimal actuation force; and
ii) it should open as wide as possible. Suitable materials for the gripper include stainless steel and titanium. The gripper size can be smaller than the diameter of 6.5 mm in the support lumen 110 in order to allow insertion and extraction of the snake robot with the gripper assembled on it. The inner faces of the gripper jaws must be machined with carefully spaced grooves in order to provide stable 3-point grasp for needles with triangular cross sections.

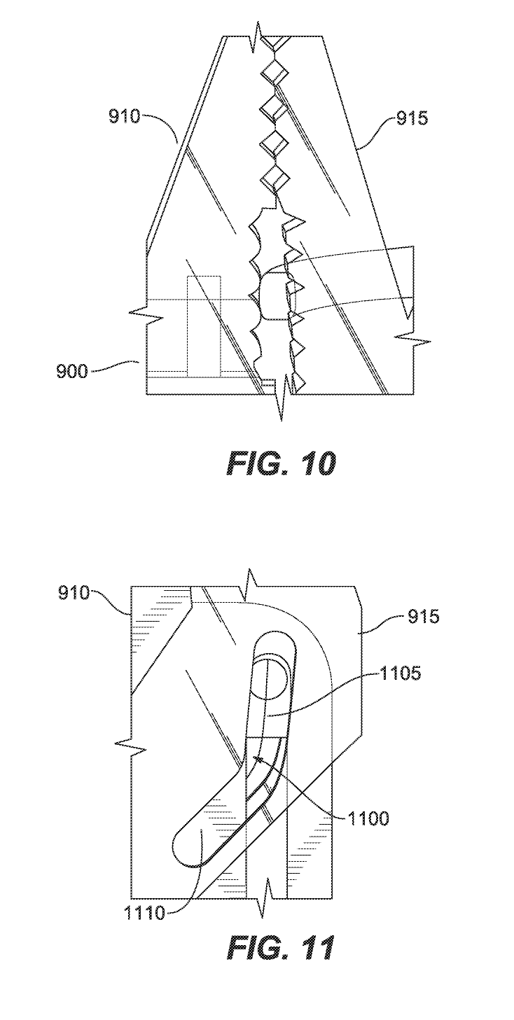

FIG. 10 is a depiction of gripper teeth of the first and second opposable end pieces 910, 915, showing different teeth for different suture sizes, according to one or more embodiments of the present disclosure. Since this gripper design only can provide enough gripping force when the jaws are almost closed, the teeth heights were assigned differently to accommodate different sutures sizes. The gripper's teeth also can be misaligned to ensure three-point contact to stabilize needles with triangular and round cross sections.

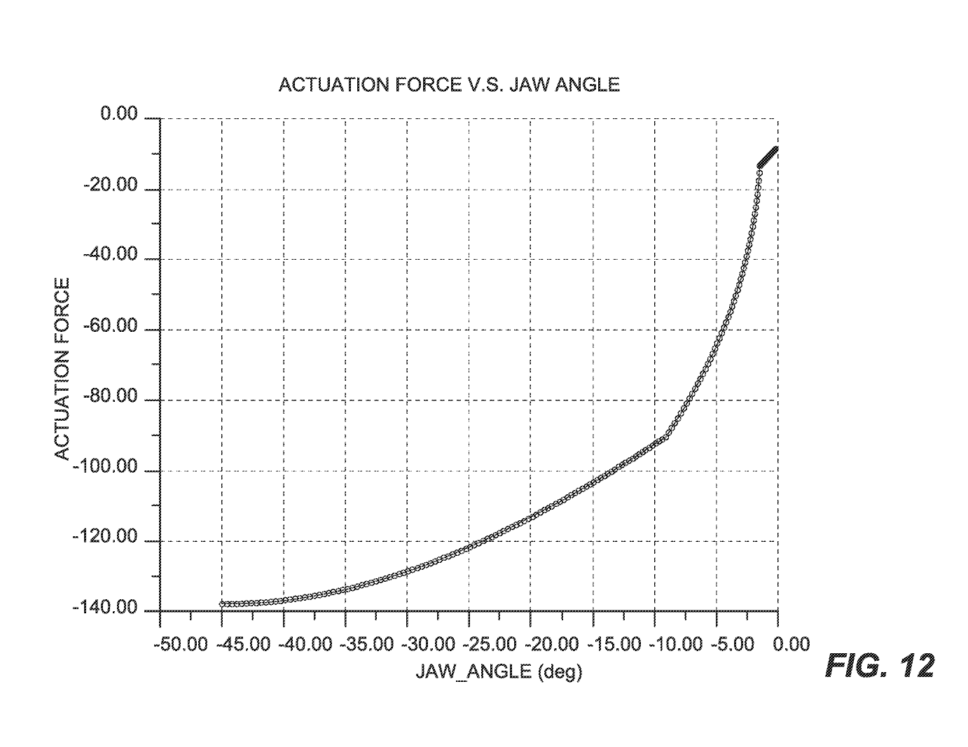

FIG. 11 is a depiction of two connected slots for both high end gripping force and wide open angle of the IREP Robot, according to one or more embodiments of the present disclosure. The first and second opposable end pieces can be slidably attached to one another. They can be connected through a first surface and a second surface of the second end piece 915 that form a slot 1100. The slot can have a first section 1105 with a first slope and a second section 1110 with a second slope. When the gripper is actuated by pushing or pulling a NiTi wire, the portion of the slot 1100 with steep slope 1105 helps generate a large gripping force by a small actuation force, while the mild slope portion 1110 opens the gripper wide over a short actuation length. This provides a gripper that has wide opening angle and a very large gripping force in a closed configuration. Simulation was conducted using the ProEngineer software program to validate the design. The results are plotted in FIG. 12. From the results, when a gripping force of 40N was maintained, the actuation force rapidly declined to around 10N, which can be easily actuated by a Ø0.4 mm NiTi Wire.

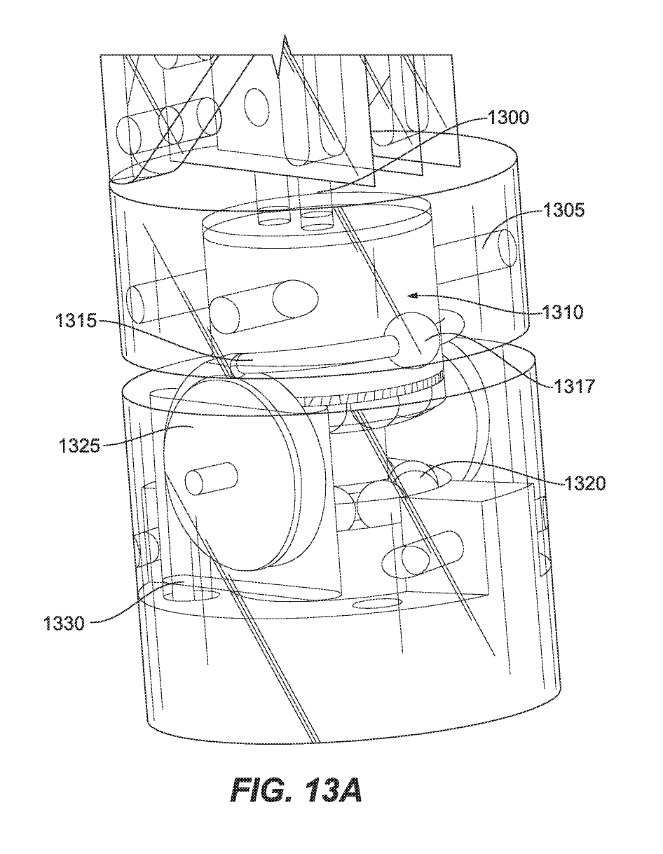

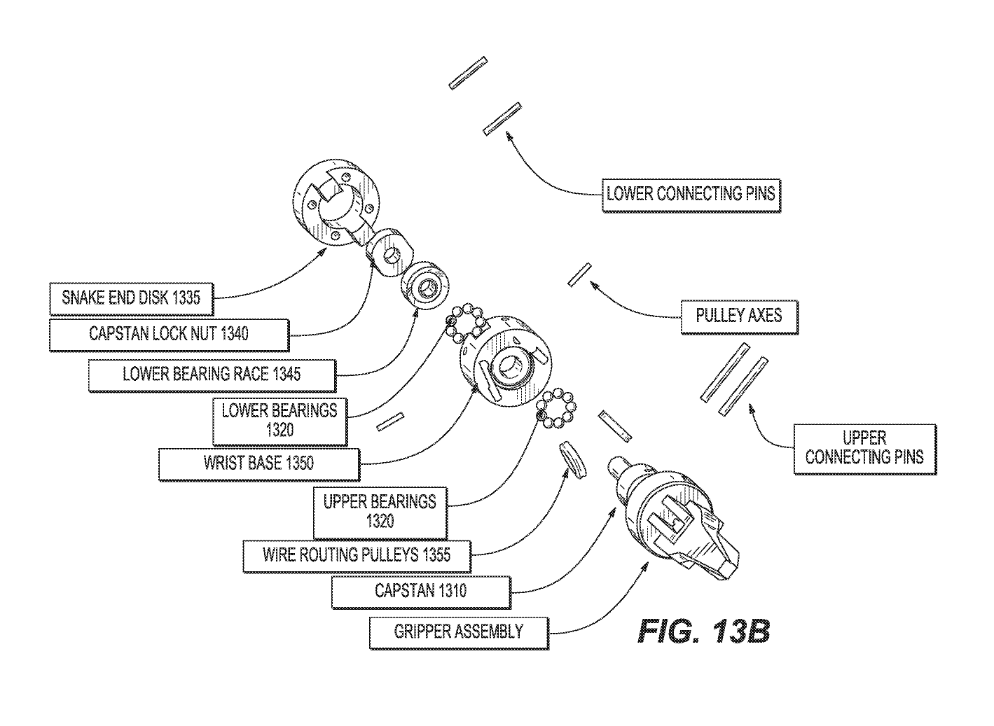

FIG. 13A is a depiction of a wrist of the IREP Robot, according to one or more embodiments of the present disclosure. The wrist includes a channel for the gripper's actuation 1300, a shear pin 1305, a capstan assembly 1310, a wire rope 1315, with a terminal 1317, a bearing assembly 1320, a pulley 1325, and a wire-rope 1330 passing through the backbone of the continuum structure. The wire-rope 1330 can be Ø0.33 mm.

FIG. 13B is an exploded view of the wrist assembly 1300. The following parts can be constructed of stainless steel, however, some biocompatible materials may be feasible for construction): snake end disk 1335, capstan lock nut 1340, lower bearing race 1345, wrist base 1350, wire routing pulleys 1355, and the capstan 1310. All shear pins and the ball bearings are constructed of hardened tool steel. The overall outside dimension of the assembly is about 6.4 mm.

The wire 1315 actively drives the wrist mechanism. The wire 1315 passes through two continuum backbones and over the capstan 1310. The terminal 1317 is connected directly to the wire rope 1315 and interfaces with the capstan 1310 as a lock mechanism such that the capstan 1310 does not slip with respect to the wire 1315. The wrist is actuated through a wire loop that passes through the super-elastic tubes of the snake arms and wraps around the capstan 1310 hinged about the longitudinal axis of the gripper. Actuation of the wire loop back and forth causes the rotation of the gripper about its longitudinal axis. A contributor to the dexterity of the IREP robot for fine manipulation tasks (including blunt dissection, dual arm manipulation and suturing) is the freedom to rotate an attached surgical end effector, such as the presented gripper, about its longitudinal axis. Previous works showed that the four degree of freedom continuum snake arm can transmit axial rotation provided that synchronous actuation of all secondary backbones is ensured by proper compensation for model imperfections. However, when the parallelogram mechanism opens and deforms the flexible stem, interaction forces can affect the transmission of the required torque of 50 mNm for suturing.

To simplify the design and control of the IREP arms, an independent single degree of freedom wrist located at the distal end of each IREP arm was chosen to meet the functional requirements, including dexterity, actuation speed and payload ability. This wrist design presents a unique challenge for robotic mechanisms of this size. Critical factors constraining the wrist design included payload, a maximum overall outside diameter defined by the external superstructure and a requirement for robustness and smooth operation in the surgical environment. The disclosed design achieves axial rotation and delivers torque via a Ø0.33 mm wire-rope running over pulleys and around a capstan arranged axially in line with the gripper. This design achieves approximately 150° of axial rotation. The distal effector platform employs a novel axial wrist design actuated by a capstan and pulley system. This wrist allows direct control of the gripper orientation about the longitudinal axis of the gripper. This added degree of freedom supports knot tying and passing sutures in very confined spaces while minimizing the required motion of the snakes. Also, this wrist allows for avoiding the requirements for very precise actuation compensation for the flexible snakes if they were used for delivering rotation along their backbone.

The actuation unit of the IREP contains three modules: a base module and two identical actuation units for two dexterous arms of the IREP (FIG. 2). The base module actuates all components of the IREP that are not interchangeable. These components include the vision module and the two five-bar parallelogram mechanisms. In addition, the base module carries all motors for the IREP and it provides gross axial motion along the axis of the IREP lumen. The actuation unit of each dexterous arm connects to the base module via a quick-connecting interface equipped with six Oldham couplings. All motors have been removed from this actuation unit in order to reduce weight and to support interchangeability of the robotic arms of the IREP. This actuation unit includes four twin lead screws for actuating the two-segment continuum robot, two lead screws to actuate the distal wrist and gripper. The distal wrist is wire-actuated and the gripper is actuated by a NiTi wire.

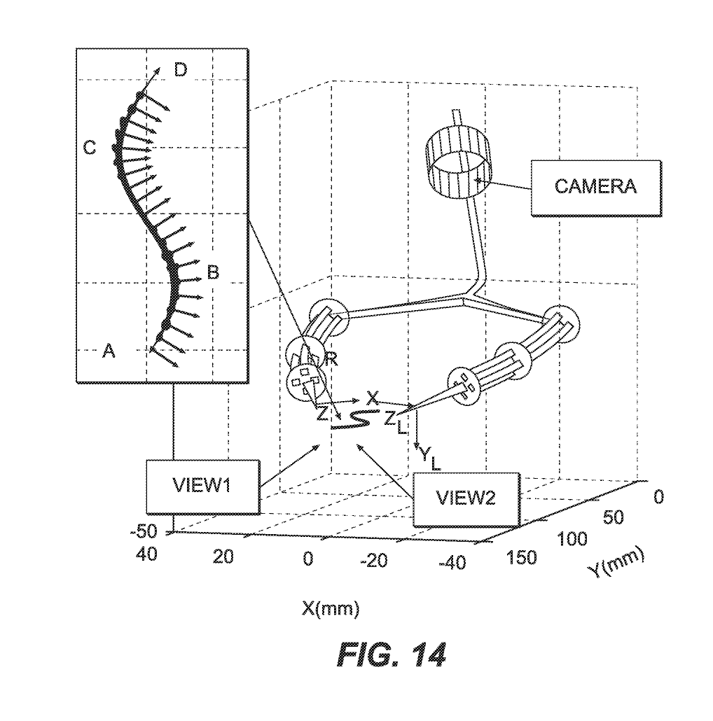

FIG. 14 depicts a dual arm suturing capability of the IREP Robot, according to one or more embodiments of the present disclosure. The dexterity of the IREP arms was verified for passing circular suturing needles at multiple locations along a sinusoidal path in the X-Y cross section of the desired workspace. The path had amplitude of 4 mm and a wave length of 40 mm. At each point along the path, the IREP inserted a ⅜ circular needle (diameter 16 mm) through 100° while holding the axis normal to its plane was tangentially aligned with the curve, shown in the inset of FIG. 14.

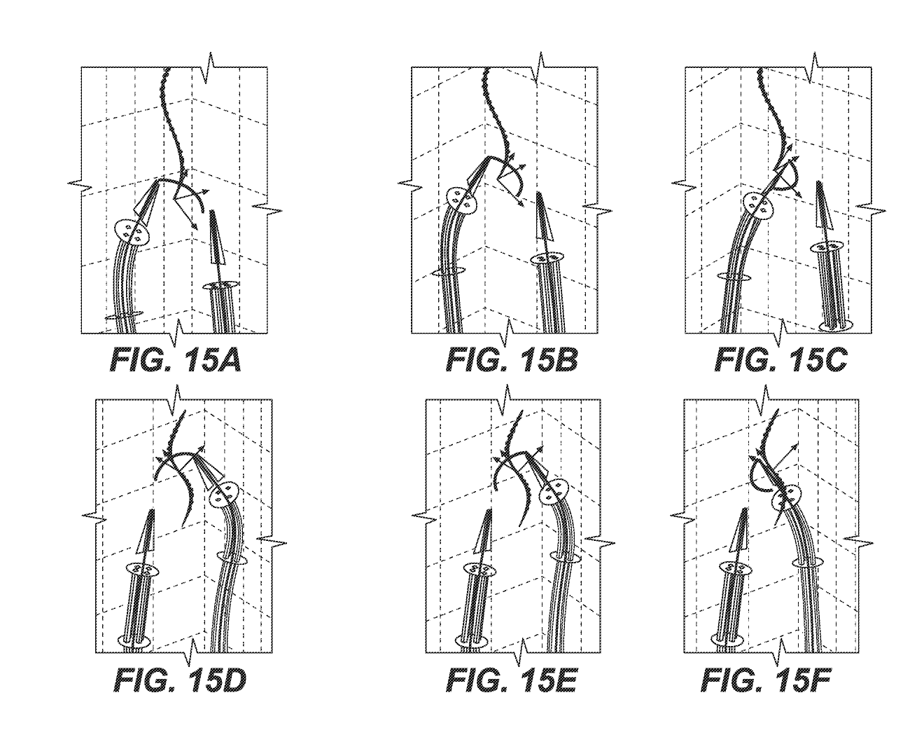

FIGS. 15A-F depicts a suturing simulation using the IREP Robot, according to one or more embodiments of the present disclosure. FIGS. 15A-C represent left hand suturing. 15D-F represent right hand suturing. The suturing arm for each segment of the curve was selected for maximum dexterity. The needle insertion motion is most easily achieved via rotated wrist joint and hence the robot is most dexterous when the wrist is aligned with given sinusoidal curve tangent. Otherwise the continuum arm will be bended in “S” shape to align of the wrist with suturing curve tangent. FIGS. 15A-C show the robot's left arm passing a circular needle at 0°, 45°, and 90° of rotation about the needle axis. FIGS. 15D-F show the right hand performing a similar task.

Though the IREP has a distal wrist, it is possible to perform the same task of passing circular sutures by using the continuum robot as a constant velocity joint to transmit rotation from its base to its gripper. This design alternative using “rotation about the central backbone” was previously explored for minimally invasive surgery of the throat. We carried out a simulation comparing the dexterity of two alternative designs of the IREP with a distal wrist or without a distal wrist. The design alternative without a distal wrist was assumed to have one degree of freedom of rotation about the base disks of each arm of the IREP in order to perform rotation about the central backbone of each arm.

In some embodiments, the IREP provides channels for energy delivery for applications such as laser surgery, cautery, radio-frequency ablation, cryosurgery, ultrasonic dissection, and new forms of energy. The IREP provides channels for sensor data and can carry sensory devices such as ultrasound probe, chemical and temperature sensors, spectral light imaging, fluorescence imaging, radioisotope imaging, or confocal microscopy. Future imaging technologies may also be deployable using this platform. The control algorithm of the IREP is capable of using information from joint level and external sensory sources for estimating the interaction forces with the tissue. This can be done using tool tip tracking (either by vision or using magnetic tracking) and by monitoring the loads on the robot arm joints.

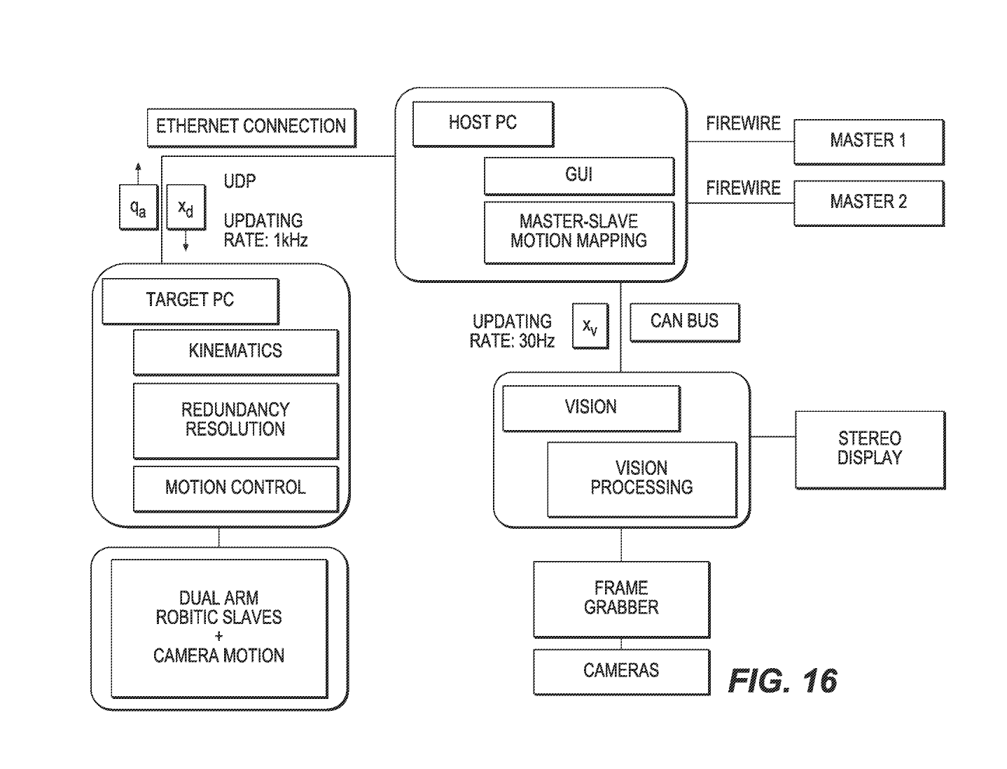

FIG. 16 is a block diagram of the control system architecture for the IREP Robot, according to one or more embodiments of the present disclosure.

The control system of the IREP robot uses a host-target environment powered by xPC Target™ from The MathWorks, Inc, which provides a rapid prototyping approach for control system setup in an open hardware architecture. Our control hierarchy is presented in FIG. 16. A GUI running on the host PC takes motion inputs from two master manipulators and then sends them down to the target PC via Ethernet connection after scaling and mapping. Target PC processes the desired motions xd of the IREP robot by solving kinematics and redundancy resolution in a 1 kHz servo control loop. A third PC running vision processing module will output the stereo display for surgeons and feed tool tracking results xv to the host PC for future motion compensation of the IREP's dual snake-like arm.

Although the invention has been described and illustrated in the foregoing illustrative embodiments, it is understood that the present disclosure has been made only by way of example, and that numerous changes in the details of implementation of the invention can be made without departing from the spirit and scope of the invention. Features of the disclosed embodiments can be combined and rearranged in various ways within the scope and spirit of the invention.

What is claimed is:

1. A foldable insertable robotic surgical device comprising:
an elongated cylindrical lumen having a distal end and a proximal end;
a plurality of flexible stems housed within the lumen prior to deployment and connected to the proximal end of the lumen;
a plurality of deployable continuum robots housed within the lumen prior to deployment, connected to the plurality of flexible stems, and each having a proximal end and a distal end;
a single degree of freedom axial wrist positioned at the distal end of each of the continuum robots;
a gripper positioned at the end of each axial wrist;
a radial extension structure spanning the proximal and distal ends of each flexible stem, wherein the radial extension structure is housed within the lumen prior to deployment and provides radial separation between the plurality of continuum robots when in a deployed state; and
a stereo vision module comprising a pair of charge coupled device (CCD) cameras housed within the lumen prior to deployment.

2. The device of claim 1, wherein the continuum robots comprise:
a plurality of disks spaced along the length of the continuum robot, comprising a base disk and an end disk;
a primary backbone having a first end and a second end, the first end affixed to the center of the base disk, the second end affixed to the center of the end disk;
four secondary backbones, spaced equidistant from each other, around the primary backbone, each of the secondary backbones having a first end and a second end, wherein the first end of the secondary backbones are affixed to the end disk and the second end of the secondary backbones are slidably attached to the base disk.

3. The device of claim 2, wherein the plurality of disks comprise a spacer disk located between the base disk and the end disk, wherein the secondary backbones are slidably attached to the spacer disk.

4. The device of claim 2, wherein the continuum robot comprises two continuum robots wherein the end disk of a first continuum robot is attached to the base disk of a second continuum robot.

5. The device of claim 2, wherein the primary and secondary backbones comprise superelastic nickel titanium.

6. The device of claim 2, wherein the primary and secondary backbones comprise concentric nickel titanium cylinders.

7. The device of claim 2, wherein the robot has a diameter of 6.4 mm or smaller.

8. The device of claim 1 comprising two continuum robots.

9. The device of claim 1, wherein the radial extension structure comprises a pivotable member secured to an actuator, wherein movement of the actuator through a first to a second position radially displaces the pivotable member.

10. The device of claim 1, wherein the radial extension structure comprises a five bar parallelogram structure.

11. The device of claim 10, wherein the five bar parallelogram structure comprises a parallelogram comprising a first bar, a second bar, a third bar, and a fourth bar, and a fifth bar configured to actuate the parallelogram.

12. The device of claim 10, wherein the five bar parallelogram structure comprises stainless steel.

13. The device of claim 1, wherein each gripper provides 40N of gripping force.

14. The device of claim 1, wherein each gripper comprises two opposable end pieces, wherein each end piece has an inner side and an outer side.

15. The device of claim 14, wherein the inner side of each gripper comprises a plurality of teeth.

16. The device of claim 15, wherein the plurality of teeth have varying heights.

17. The device of claim 14 wherein the end pieces are slidably connected through a first surface of the second end piece and a second surface of the second end piece, wherein the first surface and second surface form a slot, wherein the slot comprises a first section with a first slope and a second section with a second slope.

18. The device of claim 17, wherein the first section with the first slope corresponds to a small distance between the two opposable pieces.

19. The device of claim 17, wherein the second section with the second slope corresponds to a large distance between the two opposable pieces.

20. The device of claim 1, wherein the wrist comprises a capstan and pulley assembly.

21. The device of claim 1, wherein the wrist rotates 150 degrees.

22. The device of claim 1 comprising a plurality of flexible stems located between the proximal end of the lumen and the plurality of continuum robots.

23. The device of claim 1, wherein the lumen is rigid.

24. The device of claim 1, wherein the lumen comprises a polymer elastomer.

25. The device of claim 1, wherein the distal end of the lumen comprises a plurality of separable sidewall elements.

26. The device of claim 25, wherein the plurality of separate sidewall elements comprise a top semicircular element, having a first length, proximate to the stereo vision module and four quarter circular elements, each having a second length which is half of the first length, and two quarter circular elements located proximate to each of the flexible stems and two quarter circular elements located proximate to each of the continuum robots.

27. The device of claim 1, wherein the stereovision camera module provides images during and after insertion of the device.

28. A method of deploying a surgical tool in vivo comprising:
inserting a enclosed lumen through a single port, wherein the lumen comprises a distal portion and a proximal portion;
obtaining a visual image of the environment surrounding the distal portion of the lumen during and after insertion;
opening the distal portion of the lumen to expose a vision module and two continuum robots;
extending the vision module along the longitudinal axis of the lumen and vertically from the distal portion of the lumen;
extending the two continuum robots along the longitudinal axis of the lumen; and
separating the two continuum robots along a radial axis of the lumen using a radial extension structure.

29. A continuum robot comprising:
a plurality of disks spaced along the length of the continuum robot, comprising a base disk and an end disk;
a primary backbone having a first end and a second end, the first end affixed to the center of the base disk, the second end affixed to the center of the end disk;
four secondary backbones, spaced equidistant from each other, around the primary backbone, each of the secondary backbones having a first end and a second end, wherein the first end of the secondary backbones are affixed to the end disk and the second end of the secondary backbones are slidably attached to the base disk.

30. The continuum robot of claim 29, wherein the plurality of disks comprise a spacer disk located between the base disk and the end disk, wherein the secondary backbones are slidably attached to the spacer disk.

31. The continuum robots of claim 29 comprising two continuum robots, wherein the end disk of the first continuum robot is attached to the base disk of the second continuum robot.

32. The continuum robot of claim 29, wherein the primary and secondary backbones comprise nickel titanium.

33. The continuum robot of claim 29, wherein the primary and secondary backbones comprise concentric nickel titanium cylinders.
